(19) United States
(12) Patent Application Publication
(10) Pub. No.: US 2010/0258117 A1
(43) Pub. Date: Oct. 14, 2010

(54) Anaesthesia Breathing System

(76) Inventor: Stefan Hargasser, Munich (DE)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 12/676,309

(22) PCT Filed: Sep. 10, 2008
(86) PCT No.: PCT/EP08/07405

§ 371 Date: Jun. 21, 2010

(30) Foreign Application Priority Data
Sep. 10, 2007 (EP) 07017682.1

(51) Int. Cl. A61M 16/01; A61M 16/12; A61M 16/22

(52) U.S. Cl. 128/203.14; 128/203.12; 128/205.28; 128/205.27

(57) ABSTRACT

The invention relates to a device for recycling xenon in an anaesthesia breathing system comprising a retaining filter adapted to adsorb and desorb xenon and a first conduit connected to the retaining filter for passing a first breathing gas mixture coming from a patient and containing xenon in a first flow direction through the retaining filter, wherein at least part of the xenon remains in the retaining filter. Furthermore, the device comprises a second conduit connected to the retaining filter for passing a second breathing gas mixture in a second flow direction through the retaining filter, wherein at least part of the xenon left in the retaining filter is taken up and carried along by the second breathing gas mixture, and a supply conduit connected to the first conduit for conveying a xenon-containing anaesthetic agent into the first conduit. The invention further relates to an anaesthesia breathing system comprising the aforementioned device as well as a method for recycling xenon in an anaesthesia breathing system.

The invention relates to a device and a method for recycling anaesthetics, in particular xenon, in an anaesthesia breathing system as well as to an anaesthesia breathing system containing such a device.

The use of xenon as anaesthetic has been intensively discussed for several years. The anaesthetic effect of xenon is about 1.5 times stronger than that of nitrous oxide and had already been detected in the forties. On account of its very low blood solubility, xenon is faster eliminated by respiration than conventional anaesthetics. This is advantageous in that the anaesthetic condition of a patient can be controlled with less delay and thus more effectively. Furthermore, this entails that the patient recovers more quickly after anaesthesia and then suffers considerably less aftereffects. No detrimental side effects are known so far so that xenon obtained approval as an anaesthetic agent in Germany as early as in 2005. A xenon-containing anaesthetic agent is disclosed, for example, in WO 02/22116.

A disadvantage in the use of xenon is that xenon usually must be extracted from air in a complex method by means of fractional liquefaction, which makes it a cost-intensive raw material. Therefore, in order to improve the cost efficiency of xenon anaesthesias, possible ways of recovery have been considered. U.S. 2005/0235831 A1, for example, describes a device and method for recovering xenon from gas mixtures used in anaesthesia machines. In this method, an external recycling unit is used which recovers the xenon by means of a zeolite from the anaesthesia gas mixture already disposed of and purifies and concentrates it by use of vacuum and heat.

However, the described method is extremely complex and the device used accordingly intricate. This makes the application thereof susceptible to failure and expensive.

Hence, it is an object of the present invention to provide an improved method as well as an improved and/or more simple device for recycling xenon in anaesthesia breathing systems with which patients, such as humans and/or all kinds of animals including mammals and non-mammals, can be given artificial respiration while under anaesthesia. This object is achieved with the features of the claims. It is furthermore an object of the present invention to provide an improved method as well as an improved and/or more simple device for recycling anaesthetics in anaesthesia breathing systems.

The invention is based on the idea of providing a retaining filter in the conduit path of an anaesthesia breathing system, said retaining filter being able to collect, retain and release an anaesthetic, in particular xenon, contained in the anaesthetic gas. The anaesthetic, in particular xenon, is thus internally recycled without any additional external devices or process steps being required.

Thus, according to a preferred embodiment of the invention, the device for recycling xenon comprises a retaining filter which is adapted to adsorb and desorb xenon and a first conduit connected to the retaining filter for passing a first breathable gas mixture coming from a patient and containing xenon in a first flow direction through the retaining filter, wherein at least part of the xenon remains in the retaining filter. Furthermore, the device for recycling xenon comprises a second conduit connected to the retaining filter for passing a second breathable gas mixture in a second flow direction through the retaining filter, wherein at least part of the xenon left in the retaining filter is taken up and carried along by the second breathable gas mixture, and a supply conduit connected to the first conduit for conveying a xenon-containing anaesthetic agent into the first conduit.

Preferably, the device further comprises a sample gas outlet for feeding part of the first and/or second breathable gas mixture from the first conduit to an analysis means. The latter enables the measurement or examination of, for example, the xenon concentration in the breathable gas mixture supplied to the patient or exhaled by him/her, and thus ensures an adequate anaesthetic condition.

In order to recover the gas mixture analysed by the analysis means and in particular the xenon contained in said gas mixture, it is again fed into the system after the analysis has been performed. To this end, the device preferably comprises a connection conduit between the analysis means and the supply conduit in order to feed the breathable gas mixture emitted from the analysis means again into the first conduit.

Furthermore, a filter for adjusting the temperature and/or humidity of the first and/or second breathable gas mixture can be inserted into the first conduit. An additional carbon dioxide filter or absorber can also be provided. The filter for adjusting the temperature and/or humidity and/or the carbon dioxide filter are optionally integrated in one part together with the retaining filter.

For the appropriate administration of the anaesthesia gas mixture to the patient, the patient-side end of the first conduit preferably leads into an endotracheal tube, a larynx mask or another mask.

The retaining filter essentially comprises an organic or inorganic adsorbent which is adapted to adsorb xenon and desorb it again. At the same time, the retaining filter should be essentially permeable to oxygen, carbon dioxide and nitrogen. For this purpose, for example, activated carbon, silica, aluminium oxide, aluminium silicate, zeolite or similar materials are conceivable. The proportion of the throughflowing xenon adsorbed in the retaining filter is preferably 50 to 100%, particularly preferably 80 to 95%. As the case may be, too high a proportion can be dispensed with in order to keep the filter volume as small as possible and thus minimize the so-called dead-space volume which may unfavourably affect the patient's artificial respiration.

Since xenon typically is not used alone but administered in a manner mixed with other anaesthetics, oxygen, halogenated anaesthetics, desfluranes, sevoflurane, isoflurane, enflurane, halothane or a combination of several of these substances can be admixed to the anaesthesia gas.

Advantageously, the device further comprises a means for controlling the first and second flow directions, wherein various control modes are conceivable:

Preferably, the introduction of the xenon-containing anaesthesia gas mixture starts at the beginning of the inspiration phase, i.e. the anaesthesia gas flow is synchronized with the patient's breathing cycle. The anaesthesia gas amount applied per cycle preferably corresponds to 10 to 80% of an inspiration volume. This entails a gas flow of about 10 to 60 l/min. As an alternative to the synchronous control, a mode is possible in which a constant xenon flow is provided, which, however, reduces the efficiency of the system.

Moreover, the present invention relates to a xenon anaesthesia breathing system comprising a device for recycling xenon as described above, a means for providing the second breathable gas mixture, a means for providing the xenon-containing anaesthetic and a means for analyzing an extracted breathable gas mixture.

For the reason already mentioned, the analysis means preferably comprises a means for determining the xenon concentration in the extracted breathable gas mixture. Additionally, means for measuring further parameters, for example the concentration of other anaesthetic agents, can be provided. Moreover, in particular the determination of the oxygen concentration in the extracted breathable gas mixture is necessary.

As means for providing a breathable gas mixture, a conventional device can be used. This device may be configured, for example, as a loop that is adapted to take the filtered first breathable gas mixture, pass it through a carbon dioxide filter or absorber and provide it again. However, one-way systems are also envisaged, in which the exhaled breathable gas mixture is removed and disposed of after being filtered through the retaining filter.

The present invention further provides a device for recycling an anaesthetic in an anaesthesia breathing system. The device comprises a retaining filter that is adapted to adsorb and desorb the anaesthetic. A first conduit is connected to the retaining filter and serves the purpose of passing a first breathable gas mixture coming from a patient and containing the anaesthetic in a first flow direction through the retaining filter, wherein at least part of the anaesthetic remains in the retaining filter. Furthermore, a second conduit is connected to the retaining filter and serves the purpose of passing a second breathable gas mixture in a second flow direction towards the patient through the retaining filter, wherein at least part of the anaesthetic left in the retaining filter is taken up and carried along by the second breathable gas mixture. The device further comprises a supply conduit connected to the first conduit for conveying an anaesthetic agent containing the anaesthetic into the first conduit, wherein the first conduit further comprises a carbon dioxide filter.

The carbon dioxide filter is preferably arranged upstream of the xenon-retaining filter with respect to the first flow direction. As an alternative, the carbon dioxide filter can be arranged downstream of the xenon-retaining filter with respect to the first flow direction.

Besides, preferred embodiments of the device for recycling an anaesthetic may comprise all features described above in connection with the device for recycling xenon both individually and in combination.

Additionally, the present invention provides a method for recycling xenon from a xenon-containing first breathable gas mixture in an anaesthesia breathing system. The method comprises the following steps: Passing the first breathable gas mixture through a retaining filter in a first flow direction, wherein at least part of the xenon remains in the retaining filter, and passing an oxygen-containing second breathable gas mixture through the retaining filter in a second flow direction, wherein at least part of the xenon left in the retaining filter is discharged into the second breathable gas mixture.

When the breathable gas mixture is passed through the retaining filter, instantaneous flows between 20 and 60 l/min occur, in case of a typical respiratory volume per minute of 4 to 14 l. Since the respiratory volume per minute in young people or small animals may be considerably less, for example between 0.3 and 5 l in children, it is also conceivable to provide specific smaller retaining filters for these applications.

Furthermore, the following further steps are optionally provided: Feeding the xenon-containing anaesthetic agent into the second breathable gas mixture coming from the retaining filter, taking a sample volume from the second breathable gas mixture, measuring at least the xenon concentration of the sample volume and refeeding the measured sample volume into the second breathable gas mixture.

Furthermore, the temperature and/or humidity of the first and/or second breathable gas mixture can be adjusted.

If xenon is to be removed from the patient for reducing the xenon concentration or when terminating the anaesthetic, oxygen can be supplied instead of xenon. The volume of the supplied oxygen is preferably at least 50% of the respiratory volume per minute.

It is the aim and advantage of the above-described systems to keep xenon or the anaesthetic agent on a space as small as possible. The vaporous anaesthetics are usually distributed in the entire anaesthesia breathing system. This fact considerably contributes to an increase in the consumption of the anaesthetics. The volume of current anaesthesia breathing systems amounts to 2.5 to 3 litres. Additionally, these anaesthesia breathing systems are not completely tight so that further losses are to be expected. The systems of the present invention keep the anaesthetics (only reflexion filter) or the entire breathable gas mixture (reflexion filter and carbon dioxide absorber) in a comparably small, confined space (200 ml or 0.1 to 1 litre). It is further conceivable that the systems are combined with an additional gas supply and control which turn basically every anaesthesia breathing system/anaesthesia device into an anaesthesia device suitable for xenon. The advantage consists in the fast and simply realizable application of expensive vaporous anaesthetics. In the case of the pendulum system, the conventional anaesthesia breathing system would essentially only serve the purpose of artificial respiration, i.e. building up the pressure in the breathing system in order to pump the breathable gas mixture into the patient's lungs.

In the following, various embodiments of the present invention will be exemplarily described on the basis of the Figures.

The Figures show:

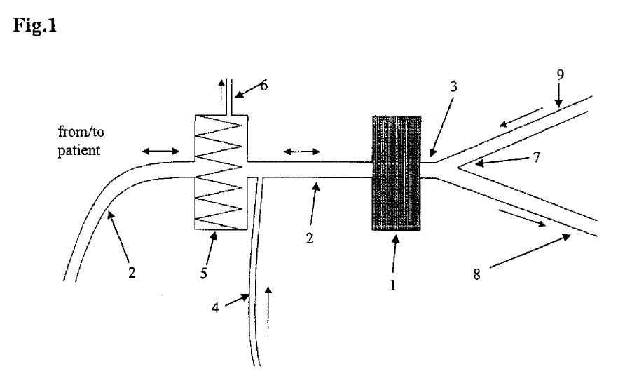

FIG. 1: a preferred embodiment of a device for recycling xenon according to the invention;

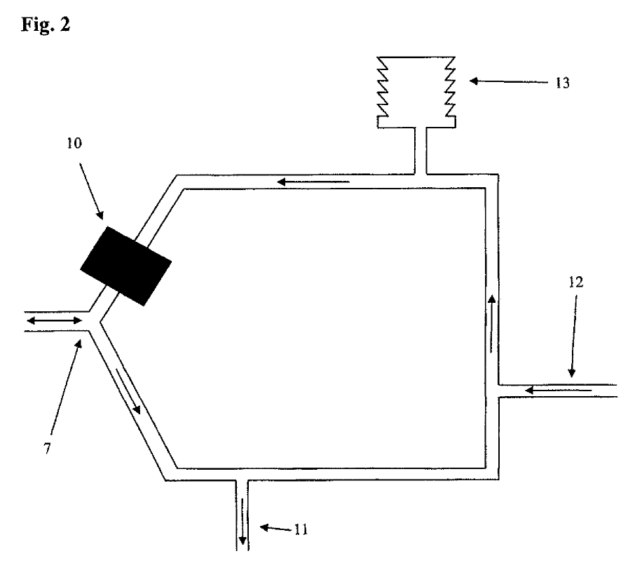

FIG. 2: a part of an anaesthesia breathing system;

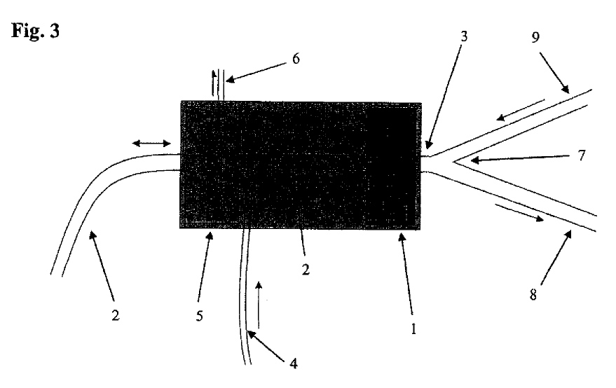

FIG. 3: a variant of the preferred embodiment of a device for recycling xenon according to the invention;

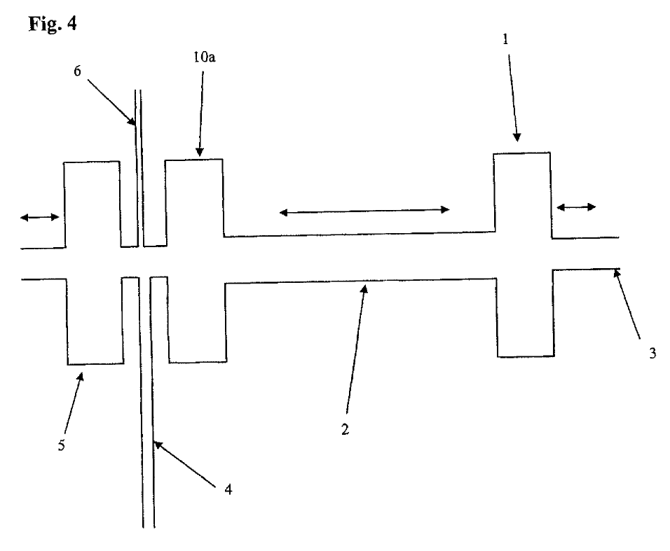

FIG. 4: a further embodiment of a device for recycling xenon according to the invention;

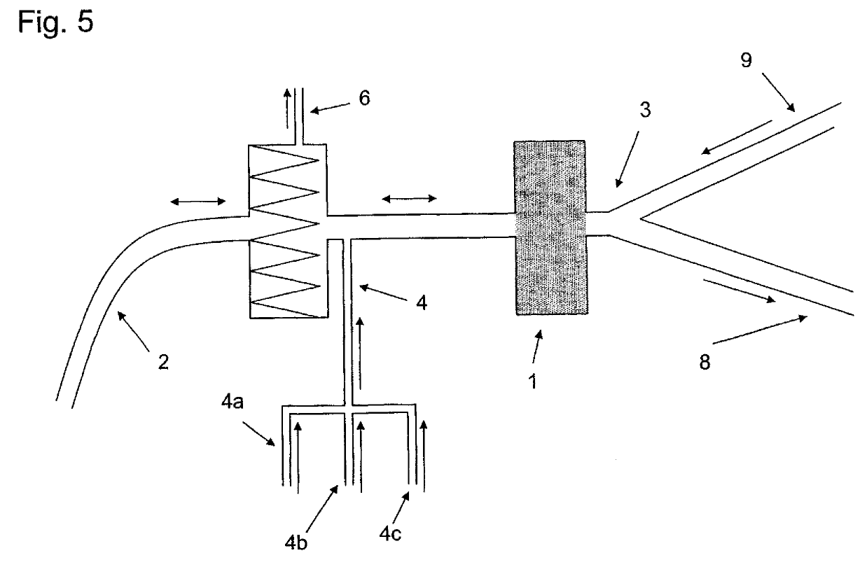

FIG. 5: an alternative of the embodiment of FIG. 1 according to the invention;

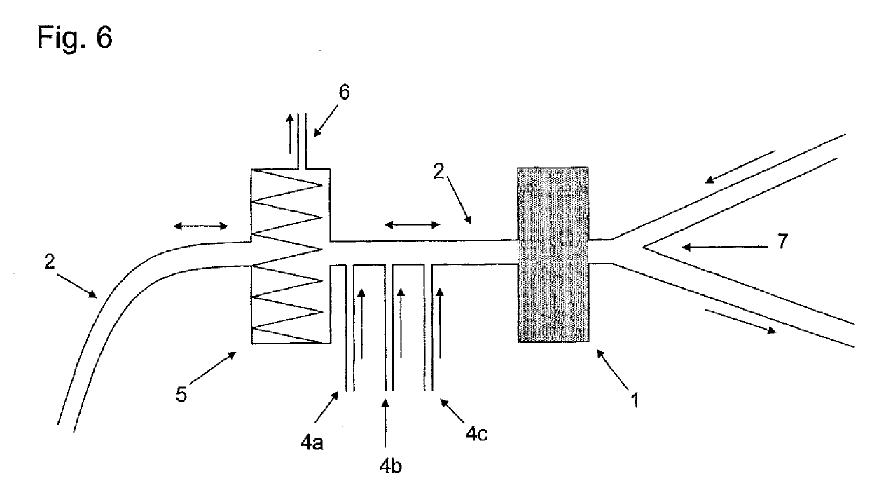

FIG. 6: a further alternative of the embodiment of FIG. 1 according to the invention;

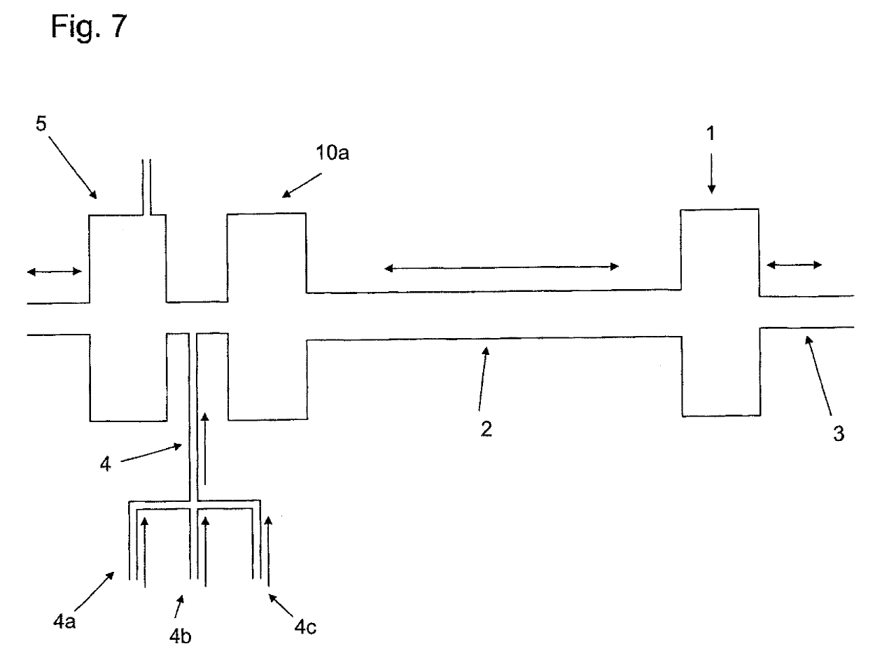

FIG. 7: an alternative of the embodiment of FIG. 4 according to the invention; and

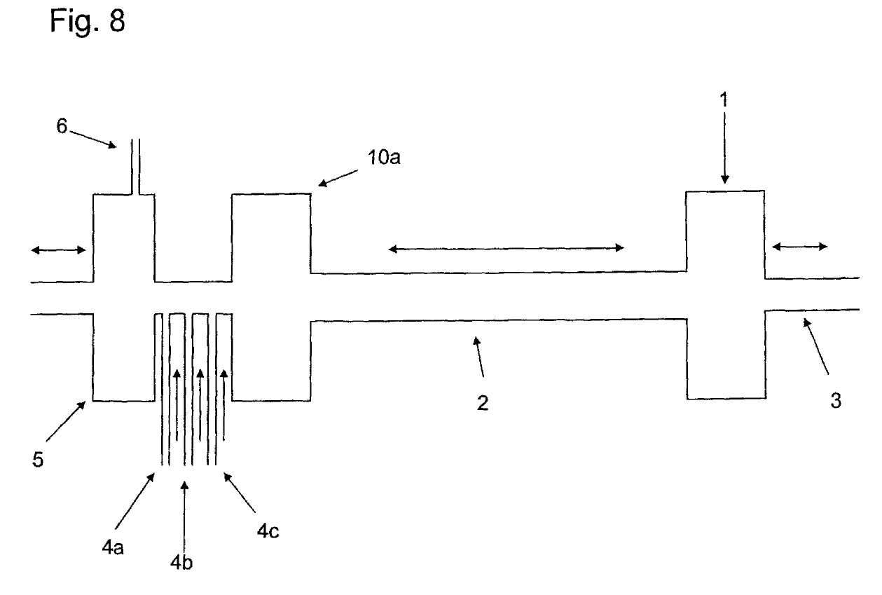

FIG. 8: a further alternative of the embodiment of FIG. 4 according to the invention.

FIG. 1 schematically illustrates a preferred embodiment of a device for recycling xenon according to the invention. This device comprises a retaining filter 1 and a first conduit 2 which connects the retaining filter 1 to a larynx mask or an endotracheal tube (not shown) at the patient-side end of the conduit 2 for the administration of an anaesthesia gas mixture to the patient and passes a xenon-containing breathable gas mixture coming from the patient through the retaining filter 1 in a first flow direction (here: towards the right side). Furthermore, a second conduit 3 is connected to the retaining filter 1 by means of which a second breathable gas mixture is passed through the retaining filter 1 in a second flow direction (here: towards the left side). An anaesthetic agent comprising at least xenon is fed into the first conduit 2 via a supply conduit 4.

Through the mask not shown, the patient inhales a gas mixture which is composed of the second breathable gas mixture supplied via the conduit 3 and the anaesthetic agent supplied via the supply conduit 4. The concentration ratios of the components can be adjusted via a control. The first breathable gas mixture exhaled again is passed via the conduit 2 through the retaining filter 1, wherein part of the xenon contained in the gas mixture is adsorbed in the retaining filter. In the next breathing cycle, this adsorbed xenon can be discharged at least partially again into the second breathable gas mixture coming from the conduit 3. It surprisingly turned out that the retaining filter shows different effects for the two flow directions on account of both the different flow rates and the respectively differing xenon concentrations of the two breathable gas mixtures: Slowly flowing xenon-rich gas preferably discharges xenon into the filter, whereas xenon-poor gas flowing faster tends to take xenon from the filter. Thus, the xenon is recycled within one breathing cycle.

The device further comprises a means 5 for exchanging heat and humidity. This means, for example, allows for partially absorbing the humidity contained in the exhaled breathable gas mixture and discharging it again into the breathable gas mixture to be inhaled. A sample outlet 6 is attached to this means for supplying part of the first and/or second breathable gas mixture from the first conduit 2 to an analysis means (not shown). The latter enables the measurement or examination of for example, the xenon concentration in the breathable gas mixture supplied to the patient or exhaled by the patient and thus ensures an adequate anaesthetic condition. The attachment of the outlet 6 to the means 5 entails good stirring of the mixture and thus the removal of a representative sample. However, another attachment is optionally also conceivable.

In order to recover the gas mixture analyzed by the analysis means and in particular the xenon contained in said gas mixture, said gas mixture is again fed into the loop after the analysis has been performed. To this end, the device preferably comprises a connection conduit (not shown) between the analysis means and the supply conduit 4 for feeding the breathable gas mixture discharged from the analysis means again into the first conduit 2.

The second conduit 3 is preferably connected to a branching piece or Y piece 7. This enables, for example, the removal and disposal of the exhausted breathable gas through the conduit 8 and the provision of fresh breathable gas through the supply conduit 9. For the purpose of a cyclic operation, however, the conduits 8 and 9 can also be connected to each other via a respective control. In this case, it is recommendable to incorporate a carbon dioxide filter 10 into the loop, e.g. between the conduits 8 and 9, for example as shown in FIG. 2.

FIG. 2 schematically illustrates an anaesthesia system to which a device for recycling xenon can be attached via the Y piece 7 as described above. FIG. 2 shows a loop in which the exhaled breathable gas mixture circulates and is provided again for inhalation in an essentially refreshed way. The mixture flows from the Y piece 7 past the outlet 11 at which excessive gas can be discharged. Fresh gas, i.e. in particular oxygen but as the case may be also an anaesthetic, can be fed into the loop through the supply conduit 12. The artificial respirator 13 serves the purpose of providing the instantaneous gas flow of 20 to 60 l/min, which is considerably greater than the continuous inflow of fresh gas through the supply conduit 12, which is typically in the order of 1 l/min. Finally, the carbon dioxide enriched by the breathing process is filtered out of the breathing gas mixture in a carbon dioxide filter 10. The corresponding volume loss is compensated for by said inflow through the supply conduit 12. When the inflow of fresh gas through the supply conduit 12 is for example increased to the amount of the respiratory volume per minute, the carbon dioxide filter is superfluous. As an alternative to supplying fresh gas through the supply conduit 12 it is also possible to introduce the fresh gas together with the anaesthetic agent through the supply conduit 4 (see FIG. 1).

In the described system, the flow direction in the individual conduits, of course, is controlled by respective valves not shown. Furthermore, the order of the components within the loop can vary. The mixture, for example, can be first passed through the carbon dioxide filter 10.

FIG. 3 schematically shows a variant of the preferred embodiment according to FIG. 1, in which the means 5 for exchanging heat and humidity as well as the retaining filter 1 are integrated in one part.

FIGS. 5 and 6 show alternatives of the embodiment of FIG. 1 according to the invention. As illustrated in FIG. 5, the supply conduit 4, for example, can comprise a branching at which two, three or more supply conduits 4a, 4b and 4c meet. Accordingly, oxygen can be introduced, for example, via the supply conduit 4a, whereas the anaesthetic agents, e.g. halogenated anaesthetics and/or xenon, are supplied via the supply conduit 4b. The oxygen and the anaesthetics then intermix to form a gas mixture or vapour at the branching. The branching preferably comprises a means for stirring the gas mixture. The third supply conduit 4c can be used to feed the breathing gas mixture discharged from the analysis means again into the first conduit 2.

Instead of the branching depicted in FIG. 5, the supply conduits 4a, 4b and 4c can also directly lead into the first conduit 2, as illustrated in FIG. 6.

It should be clear to the person skilled in the art that the embodiments shown in FIGS. 5 and 6 can be arbitrarily combined and modified. It is possible, for example, that there are only two supply conduits 4a and 4b, wherein the one is responsible for supplying oxygen and anaesthetics, the other for refeeding the breathing gas mixture discharged from the analysis means. It is also possible that there are more than three supply conduits in order to introduce, for example, different anaesthetics, such as e.g. halogenated anaesthetics and xenon, via different supply conduits. Alternatively, two supply conduits 4a and 4b can lead into one common supply conduit 4, as illustrated in FIG. 5, and additionally a supply line 4c can directly lead into the first conduit 2, as illustrated in FIG. 6.

FIG. 4 schematically illustrates a further preferred embodiment of a device for recycling xenon according to the invention, wherein said device can be used as a pendulum system. To this end, a carbon dioxide filter or absorber 10a is provided in the first conduit 2, said carbon dioxide filter or absorber being positioned upstream of the retaining filter 1 with respect to the exhalation direction. However, the carbon dioxide filer 10a can also be positioned downstream of the retaining filter 1 or configured together with it as an integral part. In contrast to the functioning of the retaining filter 1, CO₂ is only absorbed by the carbon dioxide filter 10a but not discharged again into the breathable gas mixture in the reverse flow direction. Suitable materials for the carbon dioxide filter are, for example, calcium hydroxide, sodium hydroxide, potassium hydroxide, barium hydroxide or the like, in which CO₂ is effectively bound. However, it is also possible to use a reusable carbon dioxide filter, for example a zeolite, which can be regenerated by a thermal treatment.

On account of filtering CO₂, the device can be operated as a pendulum system, i.e. the patient essentially inhales again the mixture exhaled previously into the system: The breathable gas mixture “moves in pendulum fashion” back and forth in the conduit 2. Without the carbon dioxide filter, the exhaled CO₂ would enrich in case of such an operation and lead to a CO₂ poisoning within a very short time. In this connection, the xenon retaining filter 1 prevents the diffusive loss of xenon into the artificial respiration machine. Appropriately, the volume of the first conduit 2, the carbon dioxide filter 10a and the means 5 for exchanging heat and humidity should approximately correspond to the breathing volume, i.e. preferably be in the range of 0.2 to 2.5 l, particularly preferably be in the range of 0.5 to 1.2 l. To this end, the first conduit 2 is preferably provided with a correspondingly enlarged diameter and/or an appropriate length in the area between the retaining filter 1 and the carbon dioxide filter 10a. Optionally, the retaining filter 1 and the carbon dioxide filter 10a can also be accommodated in a correspondingly dimensioned housing which then serves as conduit 2 or the two filters can be integrated into the conduit 2.

In order to replace the bound CO₂ with fresh oxygen, oxygen is provided in this embodiment through the supply conduit 4 preferably together with xenon. The pendulum operation requires that the supply of oxygen and, as the case may be, further anaesthetic agents such as desflurane etc. is close to the patient. Optionally, a further supply conduit close to the patient can also be provided. The optional sample gas outlet 6 can directly branch off from the first conduit 2, as shown in FIG. 4, or can be attached to the means 5 for exchanging heat and humidity, according to FIG. 1. Preferably, the carbon dioxide filter 10a, the retaining filter 1 as well as the means 5 for exchanging heat and humidity are accommodated in a common housing with respective connections for the supply and discharge conduits, similar to the illustration in FIG. 3.

When the carbon dioxide filter 10a is provided relatively close to the patient as shown in FIG. 4, soda lime particles perhaps escaping from it can be held by the means 5 for exchanging heat and humidity so that they cannot get into the patient's respiratory tract.

For the pendulum operation, the embodiment shown in FIG. 4 can be connected to a conventional anaesthesia system, as shown, for example, in FIG. 2. In this case, however, the use of the carbon dioxide filter 10 becomes superfluous since already the means 10a upstream of the retaining filter 1 provides for the respective filtration. Generally, however, a simple pump including a pressure control valve is sufficient instead of the loop illustrated in FIG. 2, said pump being adapted to generate the necessary pendulum flow by means of the respective sucking and pumping behaviour.

FIGS. 7 and 8 show alternatives of the embodiment from FIG. 4 according to the invention (analogously to FIGS. 5 and 6). As illustrated in FIG. 7, the supply conduit 4, for example, can exhibit a branching at which two three or more supply conduits 4a, 4b and 4c meet. Accordingly, oxygen can be introduced, for example, via supply conduit 4a, while anaesthetics, e.g. halogenated anaesthetics and/or xenon, are supplied via the supply conduit 4b. The oxygen and the anaesthetics then intermix to form a gas mixture or vapour at the branching. The branching preferably comprises a means for stirring the gas mixture. The third supply conduit 4c can be used to feed the breathable gas mixture discharged from the analysis means again into the first conduit 2.

Instead of the branching depicted in FIG. 7, the supply conduits 4a, 4b and 4c can also directly lead into the first conduit 2, as illustrated in FIG. 8.

It should be clear to the person skilled in the art that the combinations and variants discussed in connection with FIGS. 5 and 6 are also applicable to the embodiments of FIGS. 7 and 8.

Although the pendulum system shown in FIGS. 4, 7 and 8 was described above in connection with xenon as anaesthetic, it is also suitable for other anaesthetics, in particular for halogenated anaesthetics, desfluranes, sevoflurane, isoflurane, enflurane, halothane. Accordingly, the pendulum system is covered by the scope of the present invention in general and independent of the anaesthetic used.

The device of the present invention proves to be advantageous since it can be simply and cost-effectively produced and easily combined with systems already available. The device is light and compact and thus can be used in many ways and in an uncomplicated manner. The recycling is made internally, i.e. no external additional apparatuses are necessary at all for recovering the xenon.

1. A device for recycling xenon in an anaesthesia breathing system, comprising:
(a) a retaining filter adapted to adsorb and desorb xenon;
(b) a first conduit connected to the retaining filter for passing a first breathing gas mixture coming from a patient and containing xenon in a first flow direction through the retaining filter, wherein at least part of the xenon remains in the retaining filter;
(c) a second conduit connected to the retaining filter for passing a second breathing gas mixture in a second flow direction towards the patient through the retaining filter, wherein at least part of the xenon left in the retaining filter is taken up and carried along by the second breathable gas mixture; and
(d) a supply conduit connected to the first conduit for conveying a xenon-containing anaesthetic agent into the first conduit.

2. The device according to claim 1, further comprising a sample gas outlet for feeding part of the first and/or second breathable gas mixture from the first conduit to an analysis means.

3. The device according to claim 2, further comprising a connection conduit between the analysis means and the first conduit, optionally via the supply conduit, in order to feed the breathing gas mixture emitted from the analysis means again into the first conduit.

4. The device according to claim 1, wherein the first conduit comprises a filter for adjusting the temperature and/or humidity of the first and/or second breathing gas mixture.

5. The device according to claim 4, wherein the filter for adjusting the temperature and/or humidity and the retaining filter are integrated in one part or are mounted into a common housing.

6. The device according to claim 1, wherein the patient-side end of the first conduit comprises an endotracheal tube, a larynx mask or another mask.

7. The device according to claim 1, wherein the retaining filter comprises an organic or inorganic adsorbent.

8. The device according to claim 1, wherein the retaining filter comprises at least one material from the following group: activated carbon, silica, aluminium oxide, aluminium silicate, zeolite.

9. The device according to claim 1, wherein the anaesthetic agent can comprise one or more substances of the following group in addition to xenon: oxygen, halogenated anaesthetics, desfluranes, sevoflurane, isoflurane, enflurane, halothane.

10. The device according to claim 1, further comprising a means for controlling the first and second flow directions.

11. The device according to claim 1, wherein the first conduit further comprises a carbon dioxide filter.

12. The device according to claim 11, wherein the carbon dioxide filter is arranged upstream or downstream of the xenon retaining filter with respect to the first flow direction.

13. The device according to claim 11, wherein the carbon dioxide filter and the retaining filter are integrated in one part or are mounted into a common housing.

14. The device according to claim 11, wherein the carbon dioxide filter contains soda lime comprising at least one material from the following group: calcium hydroxide, sodium hydroxide, potassium hydroxide, barium hydroxide.

15. The device according to claim 11, wherein the carbon dioxide filter is a reusable filter which can be regenerated by e.g. a thermal treatment.

16. The device according to claim 11, wherein the first conduit between the carbon dioxide filter and the retaining filter is configured such that the gas volume within the first conduit including the carbon dioxide filter is between 0.2 and 2.5 l, preferably between 0.5 and 1.2 l.

17. A xenon anaesthesia breathing system comprising:
(a) a device for recycling xenon according to any claim 1;
(b) a means for providing the second breathing gas mixture;
(c) a means for providing the xenon-containing anaesthetic agent; and
(d) a means for analyzing an extracted breathing gas mixture.

18. The anaesthesia system according to claim 17, wherein the analysis means comprises a means for determining the xenon concentration in the extracted breathing gas mixture.

19. The anaesthesia system according to claim 17, wherein the means for providing the second breathing gas mixture is configured as a loop adapted to take the filtered first breathable gas mixture, pass it through a carbon dioxide filter and provide it again as second breathable gas mixture.

20. The anaesthesia system according to claim 17, further comprising a discharge conduit for removing and/or disposing of the filtered first breathable gas mixture.

21. The anaesthesia system according to claim 17, further comprising a device for providing a gas flow between 20 to 60 l/min into the second conduit.

22. A method for recycling xenon from a xenon-containing first breathable gas mixture in an anaesthesia breathing system, comprising the steps of:
(a) passing the first breathing gas mixture through a retaining filter in a first flow direction, wherein at least part of the xenon remains in the retaining filter; and
(b) passing an oxygen-containing second breathing gas mixture through the retaining filter in a second flow direction, wherein at least part of the xenon left in the retaining filter is discharged into the second breathing gas mixture.

23. The method according to claim 22, further comprising the steps of:
(a) feeding the xenon-containing anaesthetic agent into the second breathing gas mixture coming from the retaining filter;
(b) taking a sample volume from the second breathing gas mixture;
(c) measuring at least the xenon concentration of the sample volume; and
(d) refeeding the measured sample volume into the second breathing gas mixture.

24. The method according to claim 22, further comprising the step of adjusting the temperature and/or humidity of the first and/or second breathable gas mixture.

25. A device for recycling an anaesthetic in an anaesthesia breathing system, comprising:
(a) a retaining filter adapted to adsorb and desorb the anaesthetic;
(b) a first conduit connected to the retaining filter and serving the purpose of passing a first breathable gas mixture coming from a patient and containing the anaesthetic in a first flow direction through the retaining filter, wherein at least part of the anaesthetic remains in the retaining filter;
(c) a second conduit connected to the retaining filter and serving the purpose of passing a second breathable gas mixture in a second flow direction towards the patient through the retaining filter, wherein at least part of the anaesthetic left in the retaining filter is taken up and carried along by the second breathing gas mixture; and
(d) a supply conduit connected to the first conduit and serving the purpose of conveying an anaesthetic agent containing the anaesthetic into the first conduit;
wherein the first conduit further comprises a carbon dioxide filter.

26. The device according to claim 25, wherein the carbon dioxide filter is arranged upstream of the retaining filter with respect to the first flow direction.

27. The device according to claim 25, wherein the carbon dioxide filter is arranged downstream of the retaining filter with respect to the first flow direction.

28. The device according to claim 25, further comprising a sample gas outlet for supplying part of the first and/or second breathing gas mixture from the first conduit to an analysis means.

29. The device according to claim 28, further comprising a connection conduit between the analysis means and the first conduit, optionally via the supply conduit, for feeding the breathable gas mixture discharged from the analysis means again into the first conduit.

30. The device according to claim 25, wherein the first conduit comprises a filter for adjusting the temperature and/or humidity of the first and/or second breathing gas mixture.

31. The device according to claim 30, wherein the filter for adjusting the temperature and/or humidity and the retaining filter are integrated in one part or are mounted into a common housing.

32. The device according to claim 25, wherein the patient-side end of the first conduit comprises an endotracheal tube, a larynx mask or another mask.

33. The device according to claim 25, wherein the retaining filter comprises an organic or inorganic adsorbent.

34. The device according to claim 25, wherein the retaining filter comprises at least one material from the following group: activated carbon, silica, aluminium oxide, aluminium silicate, zeolite.

35. The device according to claim 25, wherein the anaesthetic can comprise one or more substances of the following group: xenon, oxygen, halogenated anaesthetics, desfluranes, sevoflurane, isoflurane, enflurane, halothane.

36. The device according to claim 25, further comprising a means for controlling the first and second flow directions.

37. The device according to claim 25, wherein the carbon dioxide filter and the retaining filter are integrated in one part or are mounted into a common housing.

38. The device according to claim 25, wherein the carbon dioxide filter contains soda lime comprising at least one material from the following group: calcium hydroxide, sodium hydroxide, potassium hydroxide, barium hydroxide.

39. The device according to claim 25, wherein the carbon dioxide filter is a reusable filter which can be regenerated by a thermal treatment.

40. The device according to claim 25, wherein the first conduit between the carbon dioxide filter and the retaining filter is configured such that the gas volume within the first conduit including the carbon dioxide filter is between 0.2 and 2.5 l, preferably between 0.5 and 1.2 l.
